(19) United States
(12) Patent Application Publication
(10) Pub. No.: US 2024/0042221 A1
(43) Pub. Date: Feb. 8, 2024

(54) THERMAL MANAGEMENT OF MEDICAL DEVICES

(71) Applicant: Shiratronics, Inc., Brooklyn Park, MN (US)

(72) Inventors: Douglas W. Brown, Shakopee, MN (US); Alan H. Smythe, White Bear Lake, MN (US); Scott A. Spadgenske, Buffalo, MN (US); Andrew J. Sowada, St. Paul, MN (US); Mitchell A. Tramp, Corcoran, MN (US); Charles W. Steidl, Edina, MN (US)

(21) Appl. No.: 18/224,184

(22) Filed: Jul. 20, 2023

Related U.S. Application Data
(60) Provisional application No. 63/370,585, filed on Aug. 5, 2022.

(51) Int. Cl. A61N 1/378; A61N 1/375

(52) U.S. Cl. CPC A61N 1/3787; A61N 1/375

(57) ABSTRACT

A rechargeable implantable neuro stimulator may include a neuro stimulation waveform generator configured to generate neuro stimulation signals. The neurostimulator may further include at least one electrode, at least one rechargeable battery configured to power the neurostimulator, and a coil for receiving power. The coil may be electrically connected to the circuitry for use in recharging the battery using the power received by the coil. The neurostimulator may further include two or more sensors configured for use in determining temperature and a controller. The controller may be configured to: control generation of neurostimulation signals from the neurostimulation waveform generator to deliver neurostimulation using the at least one electrode, perform sensor processing using the two or more sensors to determine a temperature event, and control recharging of the rechargeable battery using a recharging process including modify the recharging process to reduce heating in response to determining that the temperature event occurred.

### PRIORITY

This application claims the benefit of priority to U.S. Provisional Patent Application Ser. No. 63/370,585, filed Aug. 5, 2022, which is incorporated by reference herein in its entirety.

### TECHNICAL FIELD

This document relates generally to medical systems, and more particularly, but not by way of limitation, to systems, devices, and methods for managing medical device temperature.

### BACKGROUND

Medical devices may include devices configured to deliver a therapy, such as but not limited to an electrical or drug therapy and/or to sense physiological or functional parameters or other health-related data. The medical devices may include external wearable devices and may include implantable devices. Implantable devices configured to deliver an electrical therapy are a specific example of a medical device, and implantable neurostimulators are a specific example of implantable electrical therapy devices. A fully head-located implantable peripheral neurostimulation system, having at least two implantable devices, designed for the treatment of chronic head pain is a specific example of a system with more than one implantable device. These types of medical devices may include a rechargeable battery.

Neurostimulation systems may include a rechargeable battery, an antenna coil, and circuitry to control the neurostimulation. The systems may include one or more implantable devices configured to connect with an external unit to perform various functions such as recharging the rechargeable battery, diagnostically evaluating the implantable device(s), and programming the implantable device(s).

Heat may be generated with exposure to Magnetic Resonance Imaging (MRI) fields, electrosurgery, external defibrillation, ultrasound and electromagnetic fields. For example, heat may be generated when an implantable device is recharged using an electromagnetic field. The temperature and duration of the generated heat may be limited in an attempt to protect the patient from being harmed. For example, the standard ISO 14708-3:2017(E) discusses CEM43 dose thresholds, which is a generally accepted method to normalize the impact of temperature and time on different types of tissue for temperatures in the range of 39° C. and 57° C. By way of example and not limitation, some guidelines may indicate that an implantable neurostimulator should not generate heat where an outer surface of the implant is greater than 39° C., and that no tissue should receive a thermal dose greater than CEM43 (1 minute of exposure at 43° C.). Failure to accurately detect potentially harmful temperature events may result in false positives which may result in an implantable device unnecessarily explanted, and false negatives where a potentially harmful temperature event is not detected, such that adjacent tissue may be exposed to excessive thermal dosing.

### SUMMARY

An example (e.g., “Example 1”) of a rechargeable implantable neurostimulator, which may be configured for subcutaneous implantation and to manage heat during recharge, may include a neurostimulation waveform generator configured to generate neurostimulation signals. The neurostimulator may further include at least one electrode, at least one rechargeable battery configured to power the rechargeable implantable neurostimulator, and a coil for receiving power. The coil may be electrically connected to the circuitry for use in recharging the rechargeable battery using the power received by the coil. The neurostimulator may further include two or more sensors configured for use in determining temperature and a controller. The controller may be configured to: control generation of neurostimulation signals from the neurostimulation waveform generator to deliver neurostimulation using the at least one electrode; perform sensor processing using the two or more sensors to determine a temperature event; and control recharging of the rechargeable battery using a recharging process, including modify the recharging process to reduce heating in response to determining that the temperature event occurred.

In Example 2, the subject matter of Example 1 may optionally be configured such that the controller is configured to determine whether the temperature event occurs by measuring implant temperature from multiple sensor readings, and validating sensor measurements.

In Example 3, the subject matter of any one or more of Examples 1-2 may optionally be configured such that the controller is configured to determine a sensor fault using the sensor readings, and adjust the sensor processing to account for the sensor fault when determining the temperature event.

In Example 4, the subject matter of any one or more of Examples 1-3 may optionally be configured such that the two or more temperature sensors are positioned to detect temperature at different depths from tissue when the neurostimulator is subcutaneously implanted. Examples of such tissue may include skin, fat, connective subcutaneous tissue, aponeurosis and the like.

In Example 5, the subject matter of any one or more of Examples 1-3 may optionally be configured to further include a housing for housing at least one of the neuro stimulation waveform generator, the battery, the coil or the controller. The two or more temperature sensors may include an external temperature sensor configured to sense a temperature outside of the housing and an internal temperature sensor configured to sense a temperature inside of the housing.

In Example 6, the subject matter of any one or more of Examples 1-5 may optionally be configured such that the two or more temperature sensors include a same type of temperature sensor.

In Example 7, the subject matter of any one or more of Examples 1-5 may optionally be configured such that the two or more temperature sensors include a different type of temperature sensor.

In Example 8, the subject matter of any one or more of Examples 1-7 may optionally be configured such that the controller is configured to perform sensing processing by receiving two or more signals corresponding to the two or more sensors, and producing a fused sensor output using the two or more signals. The fused sensor output may be indicative of whether the temperature event occurred.

In Example 9, the subject matter of Example 8 may optionally be configured such that the controller is configured to denoise the received two or more signals, and apply a model to at least one of the received two or more signals to provide a virtual sensor signal used to produce the fused sensor output.

In Example 10, the subject matter of any one or more of Examples 8-9 may optionally be configured such that the controller is configured to weight the received two or more signals to produce the fused sensor output.

In Example 11, the subject matter of any one or more of Examples 8-10 may optionally be configured such that the controller is configured to perform sensor diagnostics and adjust production of the fused sensor output based on the performed sensor diagnostics.

In Example 12, the subject matter of Example 11 may optionally be configured such that the sensor diagnostics include an anomaly detection process or a fault detection process, and the sensor diagnostics further include an isolation routine to: remove one or more of the two or more signals from being used to produce the fused sensor output; or reduce a weight for one or more of the two or more signals when used to produce the fused sensor output.

In Example 13, the subject matter of any one or more of Examples 1-12 may optionally be configured such that the controller is configured to modify the recharging process in response to the temperature event by: providing a signal to an external device to stop the external device from recharging the neurostimulator; or detuning the implantable neurostimulator to reduce an amount of received energy to be dissipated as heat when the at least one rechargeable battery is fully charged.

In Example 14, the subject matter of any one or more of Examples 1-13 may optionally be configured to further include a metal can and a housing. The metal can may be configured to house the neurostimulator waveform generator and the controller. The metal can may be configured to shield the neurostimulator waveform generator and the controller from electromagnetic interference and moisture ingress. The coil may be biocompatible and not housed within the metal can. The housing may be configured to encapsulate the coil and the metal can. The housing may be non-conductive and biocompatible.

In Example 15, the subject matter of Example 14 may optionally be configured such that the housing includes silicone or an epoxy.

In Example 16, the subject matter of any one or more of Examples 14-15 may optionally be configured such that the housing is a flexible housing.

In Example 17, the subject matter of any one or more of Examples 14-16 may optionally be configured such that the housing includes a first housing portion and a second housing portion, the first housing portion encapsulates the coil and the second housing portion encapsulates the metal can, and the first and second housing portions have substantially equal footprints. Each of the first and second housing portions have a thickness, length and width. The thickness is less than the length and the width to provide each of the first and second housing portions with a substantially planar major surface. The first and second housing portions are joined such that the substantially planar major surfaces form an angle between 90 degrees and 180 degrees.

An example (e.g., “Example 18”) may include subject matter (such as a method, means for performing acts, machine readable medium including instructions that when performed by a machine cause the machine to performs acts, or an apparatus to perform) for managing heat during recharge of a rechargeable battery in an implantable medical device. The subject matter may include controlling recharging of the rechargeable battery using a recharging process, performing sensor processing using outputs from two or more sensors to determine a temperature event, and modifying the recharging process to reduce heating in response to determining that the temperature event occurred.

In Example 19, the subject matter of Example 18 may optionally be configured such that the sensor processing is performed by receiving two or more signals corresponding to the two or more sensors, and producing a fused sensor output using the two or more signals, wherein the fused sensor output is indicative of whether the temperature event occurred.

In Example 20, the subject matter of Example 19 may optionally be configured to further include denoising the received two or more signals, applying a model to at least one of the received two or more signals to provide a virtual sensor signal used to produce the fused sensor output, weighting the received two or more signals to produce the fused sensor output, and performing sensor diagnostics and adjust production of the fused sensor output based on the performed sensor diagnostics.

In Example 21, the subject matter of Example 20 may optionally be configured such that the sensor diagnostics include an isolation routine to remove one or more of the two or more signals from being used to produce the fused sensor output, or reduce a weight for one or more of the two or more signals when used to produce the fused sensor output.

In Example 22, the subject matter of any one or more of Examples 20-21 may optionally be configured to further include modifying the recharging process in response to the temperature event by providing a signal to an external device to stop the external device from recharging the neurostimulator, or detuning the implantable device to reduce an amount of received energy to be dissipated as heat when the at least one rechargeable battery is fully charged.

In Example 23, the subject matter of any one or more of Examples 18-22 may optionally be configured such that the temperature event is determined before initiating recharging of the rechargeable battery.

In Example 24, the subject matter of any one or more of Examples 18-23 may optionally be configured to further include receiving user input used to program a temperature threshold for patient comfort. The temperature event may be determined using the programmed temperature threshold.

An example (e.g., “Example 25”) may include a system that includes an external device and a rechargeable implantable neurostimulator for subcutaneous implantation and for managing heat during recharge. The implantable neurostimulator may include a neurostimulation waveform generator configured to generate neurostimulation signals, at least one electrode, at least one rechargeable battery configured to power the rechargeable implantable neurostimulator; and a coil for receiving power, wherein the coil is electrically connected to the circuitry for use in recharging the rechargeable battery using the power received by the coil. The external device may be configured to wirelessly charge and communicate with the rechargeable implantable stimulator. The rechargeable implantable neurostimulator may include at least one sensor for use in determining temperature, and the rechargeable implantable neurostimulator may include at least one sensor for use in determining temperature. The system may be configured to perform sensor processing using the two or more sensors to determine a temperature event, and control recharging of the rechargeable battery using a recharging process, including modify the recharging process to reduce heating in response to determining that the temperature event occurred.

In Example 26, the subject matter of Example 25 may optionally be configured such that the controller is configured to determine whether the temperature event occurs by measuring implant temperature from multiple sensor readings, and validating sensor measurements.

In Example 27, the subject matter of any one or more of Examples 25-26 may optionally be configured such that the controller is configured to determine a sensor fault using the sensor readings, and adjust the sensor processing to account for the sensor fault when determining the temperature event.

In Example 28, the subject matter of any one or more of Examples 25-27 may optionally be configured such that the two or more temperature sensors are positioned to detect temperature at different depths from tissue when the neurostimulator is subcutaneously implanted. Examples of such tissue may include skin, fat, connective subcutaneous tissue, aponeurosis, and the like.

In Example 29, the subject matter of any one or more of Examples 25-27 may optionally be configured to further include a housing for housing at least one of the neurostimulation waveform generator, the battery, the coil or the controller. The two or more temperature sensors may include an external temperature sensor configured to sense a temperature outside of the housing and an internal temperature sensor configured to sense a temperature inside of the housing.

In Example 30, the subject matter of any one or more of Examples 25-29 may optionally be configured such that the two or more temperature sensors include a same type of temperature sensor.

In Example 31, the subject matter of any one or more of Examples 25-29 may optionally be configured such that the two or more temperature sensors include a different type of temperature sensor.

In Example 32, the subject matter of any one or more of Examples 25-31 may optionally be configured such that the controller is configured to perform sensing processing by receiving two or more signals corresponding to the two or more sensors, and producing a fused sensor output using the two or more signals. The fused sensor output may be indicative of whether the temperature event occurred.

In Example 33, the subject matter of Example 32 may optionally be configured such that the controller is configured to denoise the received two or more signals, and apply a model to at least one of the received two or more signals to provide a virtual sensor signal used to produce the fused sensor output.

In Example 34, the subject matter of any one or more of Examples 32-33 may optionally be configured such that the controller is configured to weight the received two or more signals to produce the fused sensor output.

In Example 35, the subject matter of any one or more of Examples 32-34 may optionally be configured such that the controller is configured to perform sensor diagnostics and adjust production of the fused sensor output based on the performed sensor diagnostics.

In Example 36, the subject matter of Example 35 may optionally be configured such that the sensor diagnostics include an anomaly detection process or a fault detection process, and the sensor diagnostics further include an isolation routine to: remove one or more of the two or more signals from being used to produce the fused sensor output; or reduce a weight for one or more of the two or more signals when used to produce the fused sensor output.

In Example 37, the subject matter of any one or more of Examples 25-36 may optionally be configured such that the controller is configured to modify the recharging process in response to the temperature event by: providing a signal to an external device to stop the external device from recharging the neurostimulator; or detuning the implantable neurostimulator to reduce an amount of received energy to be dissipated as heat when the at least one rechargeable battery is fully charged.

In Example 38, the subject matter of any one or more of Examples 25-37 may optionally be configured to further include a metal can and a housing. The metal can may be configured to house the neurostimulator waveform generator and the controller. The metal can may be configured to shield the neurostimulator waveform generator and the controller from electromagnetic interference and moisture ingress. The coil may be biocompatible and not housed within the metal can. The housing may be configured to encapsulate the coil and the metal can. The housing may be non-conductive and biocompatible.

In Example 39, the subject matter of Example 38 may optionally be configured such that the housing includes silicone or an epoxy.

In Example 40, the subject matter of any one or more of Examples 38-39 may optionally be configured such that the housing is a flexible housing.

In Example 41, the subject matter of any one or more of Examples 14-16 may optionally be configured such that the housing includes a first housing portion and a second housing portion, the first housing portion encapsulates the coil and the second housing portion encapsulates the metal can, and the first and second housing portions have substantially equal footprints. Each of the first and second housing portions have a thickness, length and width. The thickness is less than the length and the width to provide each of the first and second housing portions with a substantially planar major surface. The first and second housing portions are joined such that the substantially planar major surfaces form an angle between 90 degrees and 180 degrees.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIGS. 1A-1B illustrate a system that includes implantable device(s) and an external device configured for use to communicate with and charge the implantable device(s).

FIG. 2A depicts two implanted devices with leads to cover both sides of the head with one on the left side of the head and the other on the right side of the head, and FIG. 2B illustrates a charging/communication headset disposed about the cranium.

FIG. 3 illustrates, by way of example and not limitation, an embodiment of an external charging system.

FIGS. 4A-4C illustrate, by way of example and not limitation, an implantable device.

FIGS. 5A-5B illustrate a headset 503, similar to the headset illustrated in FIGS. 1A-1B.

FIGS. 6A-6C illustrate, by way of example and not limitation, some components of an implantable device, including a conductive enclosure for encasing electronic circuitry.

FIG. 7 illustrates, by way of example and not limitation, a diagram of a rechargeable implantable neurostimulator.

FIG. 8 illustrates, by way of example and not limitation, sensor processing for temperature events performed using the controller illustrated in FIG. 7.

FIG. 9 illustrates, by way of example and not limitation, a sensing processing embodiment that weights sensor signals and uses fault diagnostics to adjust the weighted sensor signals.

FIG. 10 illustrates, by way of example and not limitation, anomaly detection, fault detection and fault isolation used to weight sensor signals.

FIG. 11 illustrates, by way of example and not limitation, a block diagram of an implantable device.

FIG. 12 illustrates, by way of example and not limitation, an embodiment of an implantable device.

FIG. 13 illustrates, by way of example and not limitation, a method for managing heat during recharge of a rechargeable implantable neurostimulator.

FIG. 14 illustrates, by way of example and not limitation, a method for performing sensor processing using outputs from two or more sensors

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

The present subject matter relates to thermal management for medical devices such as implantable medical devices. Such thermal management may protect patients from heat that potentially may be generated by an implantable device in the presence of MRI fields, electrosurgery, external defibrillation, ultrasound and electromagnetic fields. More particularly, the present subject may include passive thermal management and/or active thermal management. Passive thermal management relates to the material used to encapsulate the implant to reduce the heat generated on an implantable device during recharge. Active thermal management relates to the capability of providing reliable and fault-tolerant control for thermal management of implantable devices during recharge.

Challenges for implantable medical device design include limiting the amount of heat generated at the surface of the implant, and shielding internal circuitry from electromagnetic field interference (EMI). Previous neuromodulators rely on metals and/or alloys, such as stainless steel, titanium and its alloys and cobalt alloys, to house the device. However, these materials generate excess heating on the surface of the implant during recharging. Wireless recharging uses a transmit coil (e.g., external device coil) to generate a magnetic field, and a receive coil (e.g., implantable device coil) in the presence of the magnetic field generates a voltage. When the housing of the implantable device is metal or other conductor, the magnetic field induces eddy currents on the surface of the implant which may generate heat. The heat generated by the eddy currents induced by the magnetic field may increase the applied thermal dose to the surrounding tissue.

To overcome these challenges, the present subject matter uses a non-conductive bio-compatible material to encapsulate the implant and the implant contains an electrically isolated metal enclosure to mitigate EMI of internal circuitry. Embodiments of the present subject may reduce the amount of heat generated due to eddy currents by reducing the total metallic surface area. Instead of the outer surface of the implant being comprised of metal, the outer surface is comprised of a non-conductive material. Within the non-conductive material, a smaller metal enclosure houses the internal electronics such as an application specific integrated circuit (ASIC) and microcontroller unit (MCU). This metal enclosure provides electrical shielding for battery and electronics as well as provides a hermetic seal to protect against moisture ingress. The battery and electronics within the can are electrically isolated from the metal can. Whereas prior devices were completely housed by a metal or other conducting material, the present subject matter only uses metal to shield some electronics. Thus, for the same size implant, the amount of conducting surface is reduced such that the amount of surface heating caused by eddy currents is reduced. Furthermore, the non-conductive material of the housing may insulate the tissue from the heat generated by eddy currents in the smaller metal enclosure. By way of example, the implant may be over-molded with a mildly flexible non-conductive material, such as silicone, epoxy, or other insulative material. This non-conductive material minimizes or otherwise reduces the amount of heat generated at the surface of the implant during recharging of the implant. The encapsulation of the implant in non-conductive insulative material provides a reduction in temperature rise during heat generation associated with device battery charging.

In addition to the conductive (e.g., metal) enclosure, the coil may also be a source of heat in an implantable device. Various embodiments may detune the implant receiving circuit and receive coil inhibits the transfer of energy from the external charger thereby mitigating the overall heat generated due to the ohmic loses in the receive coil. Additionally or alternatively, during concurrent implant recharging of two or more devices and when one implant is over-temperature, the charger can respond to an event in which one of the devices is over a temperature threshold by stopping charging of the device that is over the temperature threshold while continuing to charge the other implant as discussed in U.S. Provisional Application No. (Attorney Docket 5467.017PRV), entitled “Multi Implantable Device Communication and Charging System,” and filed on the same date as the present application, which is herein incorporated by reference in its entirety. Active thermal management may include the ability to isolate recharging of implants in response to over-temperature events which allows other devices which are not experiencing a temperature event to continue with their charging while temporarily stopping or reducing the charge transfer to the device that is experiencing the temperature event.

Active thermal management may include the use of two or more internal implant temperature sensors for thermal management of the implantable device during recharging of the implantable device. The temperature sensors may be located on two or more planes of the device (e.g., front side and back side to provide measurement at both surface of implant, or at different depths (e.g., different planes corresponding to different depths beneath the skin when the device is subcutaneously implanted beneath the skin and over the cranium). These multiple sensors may be used to provide temperature information at deeper locations as well as ability to provide a thermal gradient to estimate surface temperature.

Another challenge involves accurately determining over-temperature faults for the device. For example, the use of a single sensor with no redundancy can result in single-fault failures due to false-negatives where the implant exceeds the maximum thermal dose. Conversely, false positives may require the implant be explanted due to re-occurring false alarms. Active thermal management may include the use of multiple temperature sensors, virtual sensors, or indirect thermal measurements to provide fault tolerance. Multiple sensor readings may be used to measure implant temperature. Sensor measurements may be validated in in real-time to identify whether a sensor reading is valid or faulty. A faulty sensor reading may be isolated from the implant temperature measurement. This information may be included in data retrieved or sent to a clinician. Multiple temperature sensors may include at least one temperature sensor on or in an external device (e.g., charger) and at least one temperature sensor on or in an implantable device.

As an example of active thermal management, various embodiments of the present subject matter may use sensor fusion and fault diagnostics to ensure continuous operation in the event of a fault (e.g., fault tolerance). Examples of fault diagnostics include anomaly detection, fault identification and isolation. Such fault diagnostics along with sensor fusion may be used to ensure continuous operation in the event of a fault.

The ability to continue operating normally despite the occurrence of one or more faults is known as “fault tolerance”. Various embodiments may use a routine that converts a sensor measurement to a different scientific unit of measure (referred to as a “virtual sensor”). Various embodiments may implement denoising, which is a process of removing noise from a signal. Before data is used to make decisions it is run through preprocessing routines to denoise the data. Denoising includes the application of signal processing routines through the application of band-pass filters, median filters, and autoregressive-moving average models to remove unwanted portions of the signal or noise. Various embodiments may use sensor fusion, which refers to the use of two or more sensor measurements (which may include virtual sensors) to generate an output resulting in less uncertainty when compared to the uncertainty of any one individual sensor. For example, fused sensor outputs may be computed continuously based on persistently stored weighted average coefficients. By way of example and not limitation, a simple implementation of sensor fusion is averaging redundant sensor outputs. Enhanced sensor fusion may employ statistical-based methods such as Bayesian inferencing or Kalman Filtering when the uncertainty of each sensor is known a-priori. Similar uncertainty information can be obtained as part of the sensor fault detection and severity.

In an embodiment having two or more sensors, only one sensor may normally be used to detect the temperature event when there is no detected fault. If a fault occurs, the sensing may use one or more of the other sensors in its place. In an embodiment having two or more sensors, a weighted average of each sensor may be used to detect the temperature event.

Some embodiments may implement fault tolerance using fault diagnostics. Fault diagnostics is a process of detecting a fault and isolating the source of the fault. For example, a backup sensor may be used if one or more sensors are identified as being faulty. In some embodiments, a weighted average of each sensor may be used such that the contribution of each sensor is dependent on the fault severity. Changing a weighted average between two sensors from 1 to 0 and 0 to 1 is equivalent to not using the sensor (e.g., switching to a backup sensor).

Examples of fault diagnostics may include anomaly detection, which identifies rare events, items, or observations which are suspicious because they differ significantly from standard behaviors or patterns. Anomaly detection may be periodically performed in the background to continuously monitor for occurrences of anomalies. Fault detection and isolation may only run when necessary to reduce computation time and overhead. Anomalies may occur as persistent or intermittent events. Anomalies may be assessed through a combination of data driven methods and physics-based modeling. Data-driven anomaly detection methods may be based on expected behavior gained from historical data and a-priori information. Historical data may provide a baseline of expected behavior derived from previously collected data over a range of operating conditions using statistical measures such as computed means, standard deviations, outliers, repeated values, missing values, and noise. A-priori information may be acquired through expected behavior that can be derived from specifications such as operating limits and response times. Examples of data-driven anomaly detection may include, but is not limited to, the detection of: an occurrence rate of outliers, a trend (e.g., positive or negative) in the mean occurs over time, spurious noise, or sensor outputs that deviate from other redundant sensors.

Model-based anomaly detection methods may be based on the use of physical models used to compare two or more inputs. For example, a thermal model may be used to relate the output of multiple sensors using physical and thermal conductivity characteristics between sensors. Statistically significant deviations from the predicted relationship may be indicative of an anomaly. A positive confirmation a fault has occurred may be referred to as a fault detection. Fault detection routines may be triggered when an anomaly is observed. Faults are anomalies that are persistent and demonstrate statistical significance with respect to a Type I error (probability of false detection) and Type II error (probability of misdetection). Hypothesis testing may be used to determine the statistical significance of the anomaly. Parameterized Probability distributions such as Gaussian, Poisson, and Log-Normal distributions are determined a-priori from characterization data. Non-Parameterized probability distributions can also be used that are estimated in real-time using numerical methods such as recursive Monte Carlo filters.

Fault isolation relates to an assessment of the fault based on the relative contribution of each sensor. Fault isolation routines may be triggered when a fault has been declared during fault detection. The fault severity may be categorical or numerical. For example, a fault severity may correspond to a numerical scale from 0 to 1 where 0 corresponds to no fault and 1 corresponds to a fault. Numerical representations can be naturally expressed as a function of the Type I error and type II error computed during Sensor Fault Detection. Fault severity may also be expressed categorically. For example, if the probability of a fault detection exceeds a given threshold, the sensor may be characterized as FAULT and characterized as GOOD otherwise. Bayesian inferencing can also be used to categorically identify the likelihood of the source of the sensor fault.

The output of the fault isolation routine may be used to update the weighted average coefficients based on the fault severity corresponding to each sensor. Persistent memory may be used to store weighted average coefficients that are initially determined a-priori (initial condition). These coefficients may be updated by the fault isolation routine when a fault has been declared as a result of the fault diagnostics module.

Temperature measurements may be used to end the recharge session only for safety purposes. Various embodiments of the present subject matter can perform concurrent implant recharging (two devices) to simultaneously charge each device, but still has independent control of the charging so that one of the devices can still be charged if the other has a temperature event. This improves overall performance by reducing the overall recharge time. Single-fault tolerance for the implant temperature measurement allows the implant to be used in normal operation without the risk of increasing the applied thermal dose during implant recharge and reduces the likelihood of false negatives (failure to identify over temperature events) and false positives (resulting in nuisance alarms increasing the likelihood of an explant event). Detuning the implant receiving circuit and receive coil reduces overall heat-generation of the implant by lowering the ohmic loses generated on the coil in the presence of an externally applied magnetic field.

By way of example, this disclosure discusses a fully head located implantable peripheral neurostimulation system designed for the treatment of chronic head pain. The system may be configured to provide neurostimulation therapy for chronic head pain, including chronic head pain caused by migraine and other headaches, as well as chronic head pain due other etiologies. For example, the system may be used to treat chronic head and/or face pain of multiple etiologies, including migraine headaches; and other primary headaches, including cluster headaches, hemicrania continua headaches, tension type headaches, chronic daily headaches, transformed migraine headaches; further including secondary headaches, such as cervicogenic headaches and other secondary musculoskeletal headaches; including neuropathic head and/or face pain, nociceptive head and/or face pain, and/or sympathetic related head and/or face pain; including greater occipital neuralgia, as well as the other various occipital neuralgias, supraorbital neuralgia, auriculotemporal neuralgia, infraorbital neuralgia, and other trigeminal neuralgias, and other head and face neuralgias.

The system may include two implantable devices bilaterally implanted on the right and left sides of the patient's head. However, the present subject matter is not limited to such systems, as those of ordinary skill in the art would understand, upon reading and comprehending this disclosure, how to implement the teachings herein with other systems such as, but not limited to, two or more rechargeable medical devices that are implantable or wearable.

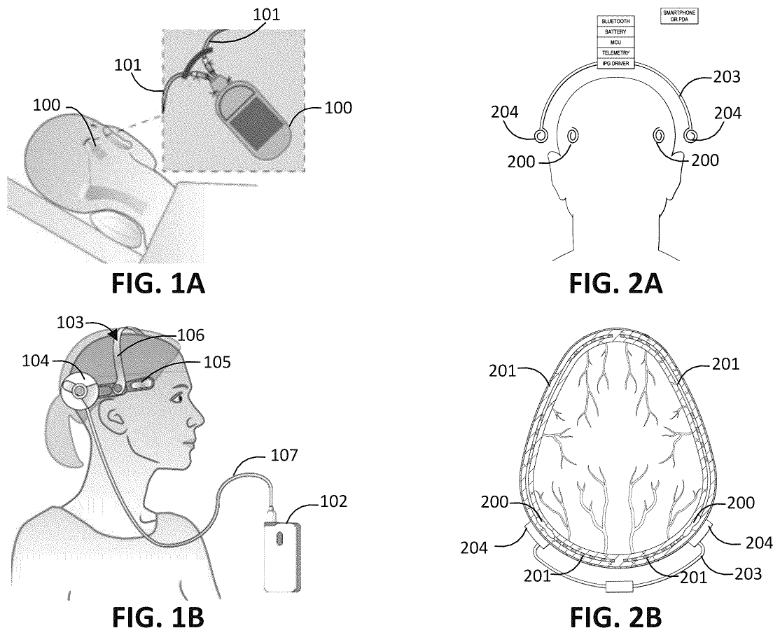

FIGS. 1A-1B illustrate a system that includes implantable device(s) and an external device configured for use to communicate with and charge the implantable device(s). FIG. 1A illustrates an implantable device 100 implanted beneath the skin and over a patient's cranium. The device 100 is illustrated as being implanted behind and above the ear. The implantable device may include one or more leads 101 that may be subcutaneously tunneled to a desired neural target. Each lead may include one or more electrodes. The number of electrodes and spacing may be such as to provide therapeutic stimulation over any one or any combination of the supraorbital, parietal, and occipital region substantially simultaneously. The implantable device 100 may be configured to independently control each electrode to determine whether the electrode will be inactive or configured as an electrode or an anode. One or more electrodes on the lead(s) may be configured to function as an anode, and one or more electrodes on the lead may be configured to function as a cathode. For example, bipolar neuromodulation may be delivered using one or more anodes and one or more cathodes on the lead(s). A clinician may program the electrode configurations to provide a neuromodulation field that captures a desired neural target for the therapy.

FIG. 1B illustrates an external device 102 and headset 103 configured for use to communicate with and/or charge the implantable device(s) 100. The headset 103 may include an external coil 104, and the headset 103 may be configured to position the external coil over an implantable device. For example, the headset 103 may include an adjustable frame 105 on each side of the head that can rotate about a point on a main headset frame 106, and may be configured to provide addition degrees of motion (e.g., sliding or pivoting motion) with respect to the main headset frame 106. These adjustable frames may be used to position the external coils 104 over the implantable devices 100 when the main headset frame 106 is worn. The external device 102 may be electrically connected to the external coil 103 via a cable 107. In some embodiments, the external device 102 may be wirelessly connected to the headset 103. The headset may be configured to wirelessly receive power from the external device and to transfer power from the external coil to the implanted device(s).

FIG. 2A depicts two implanted devices 200 with leads 201 to cover both sides of the head with one on the left side of the head and the other on the right side of the head, and FIG. 2B illustrates a charging/communication headset 203 disposed about the cranium. The headset 203 may include right and left coupling coil enclosures, respectively that contain coils for coupling to the respective coils in the implants. The coil enclosures interface with a main charger/processor body which contains processor circuitry and batteries for both charging the internal battery in the implantable devices 200 and also communicating with the implanted devices. Thus, in operation, when a patient desires to charge their implanted devices 200, all that is necessary for some embodiments is to place the headset about the cranium with the coils 204 in close proximity to the respective implanted devices 200. In some embodiments, such placement may automatically initiate charging; whereas in other embodiments, the user may initiate charging using an external device. When the headset 203 is worn by a patient, the headset coils (transmit. coils) 204 are placed in proximity to the corresponding receive coil in each respective body-implanted implantable device 200. As illustrated, the headset 203 may include an implantable device driver, telemetry circuitry, a controller or MCU, a battery, and a Bluetooth wireless interface. The headset 203 may also communicate with a personal device such as a smartphone or tablet (e.g., via the Bluetooth interface), for monitoring and/or programming operation of the two implantable devices.

The implantable device may include a rechargeable battery, an antenna (e.g., coil), and an ASIC, along with the necessary internal wire connections amongst these related components, as well as to the incoming lead internal wires. These individual components may be encased in a can made of a medical grade metal, which may be encased by plastic cover. The battery may be connected to the ASIC via a connection that is flexible. The overall enclosure for the battery, antenna and ASIC may have a very low flat profile with two lobes, one lobe for housing the ASIC and one lobe for housing the battery. The antenna may be housed in either of the lobes or in both lobes. The use of the two lobes and the flexible connection between the ASIC and the battery allows the implanted device to conform to the shape of the human cranium when subcutaneously implanted without securing such to any underlying structure with an external fixator.

The ASIC and lead may be configured to independently drive the electrodes using a neuromodulation signal in accordance with a predetermined program. The programmed stimulation may be defined using parameters such as one or more pulse amplitudes, one or more pulse widths and one or more pulse frequencies. Other parameters may be used for other defined waveforms, which may but does not necessarily use rectilinear pulse shapes. Once the program is loaded and initiated, a state machine may execute the particular programs to provide the necessary therapeutic stimulation. The ASIC may have memory and be configured for communication and for charge control when charging a battery. Each of the set of wires and interface with the ASIC such that the ASIC individually controls each of the wires in the particular bundle of wires. Thus, each electrode may be individually controlled. Each electrode may be individually turned off, or as noted above, each electrode can be designated as an anode or a cathode. During a charging operation, the implanted device is interfaced with an external charging unit via the antenna (e.g., coil) which is coupled to a similar antenna (e.g., coil) in the external charging unit. Power management involves controlling the amount of charge delivered to the battery, the charging rate thereof and protecting the battery from being overcharged.

The ASIC may be capable of communicating with an external unit, typically part of the external charging unit, to exchange information. Thus, configuration information can be downloaded to the ASIC and status information can be retrieved. Although not illustrated herein, a headset or the like may be provided for such external charging/communication operation.

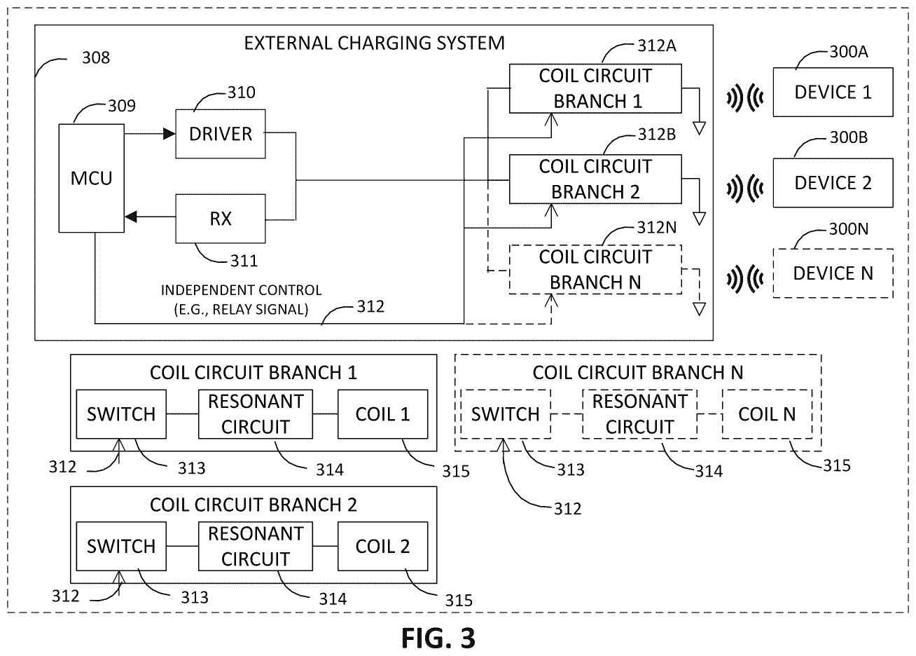

FIG. 3 illustrates, by way of example and not limitation, an embodiment of an external charging system. The external charging system 308 is configured to wirelessly charge at least two devices, and may be configured to wireless charge more than two devices (e.g., N devices), illustrated as Device 1 300A, Device 2 300B and Device N (300N). The external charging system 308 may take the form of the headset 103, 203 illustrated in FIGS. 1B, 2A-2B. The external charging system 308 may include a controller 309 (e.g., microcontroller or MCU), a driver circuit, or driver, 310 and a receiver circuit, or receiver, 311. The external charging system 308 may further include a first coil circuit branch 312A and a second coil circuit branch 312B. If configured to wirelessly charge N devices, the external charging system 308 may include N coil circuit branches, including an Nth coil circuit branch 312N. The coil circuit branches are connected in parallel. The devices may not be “paired” to the coil circuit branches, but rather any of the coil circuit branches may be positioned and used to wireless charge any of the devices. That is, they may be interchangeable and do not have to be paired. Each of the circuit branches 312A-312N may be selectively connected to the driver 310 and receiver 311 via a control signal 312 from the controller 309. For example, each coil circuit branch may include a switch 313, a resonant circuit 314 and a coil 315 connected in series. The switch 313 is configured to respond to the control signal 312 from the controller 309 to selectively connect the coil 315 for the circuit branch to the driver 310 and receiver 311. Thus, the controller 309 and switches cooperate to independently connect or disconnect the coil from the driver 310 or receiver 311. By way of example, the switch 313 may be a relay switch. Other switch examples include transistors, TRIACs or other solid state switch devices. Thus, the external charging system 308 is capable of individually controlling which of the coils in the parallel-connected, coil circuit branches is connected. Thus, the external charging system is capable of simultaneously using all coil circuit branches to concurrently charge all corresponding implantable devices, and is also capable of individually disconnecting coils to stop charging to individual ones of the devices if the device is experiencing a temperature event. The resonant circuit 314 creates a resonant frequency close to the drive frequency to maximize efficiency, and may also provide matching impedance to a cable such as a 75 Ohm cable.

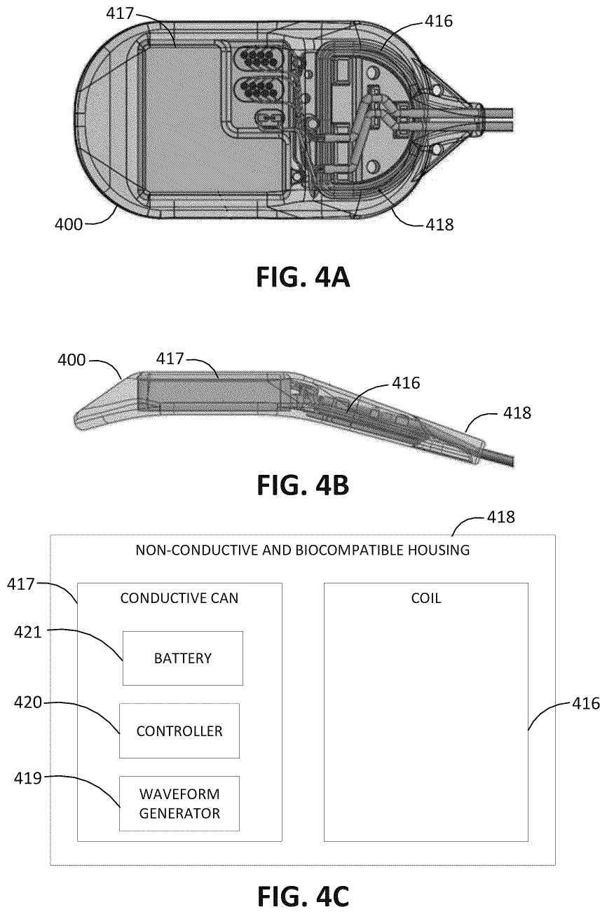

FIGS. 4A-4C illustrate, by way of example and not limitation, an implantable device. The illustrated device is configured for subcutaneous implantation, and more particularly is configured for subcutaneous implantation over a cranium. The device 400 includes a coil 416 used for communication and charging, a conductive enclosure 417 (e.g., metal can) for electronics, and a non-conductive and biocompatible coating 418 that encases the conductive enclosure 417 and the coil 416. The electronics within the conductive enclosure may include a neurostimulator waveform generator 419, a controller 420 and a battery 421. A microcontroller unit (MCU) and application specific integrated circuit (ASIC) may provide the controller and the neurostimulator waveform generator functions. The MCU may contribute to one or both of the controller and the neurostimulator waveform generator functions. The ASIC may contribute to one or both of the controller and the neurostimulator waveform generator functions. The MCU and ASIC may be encased within the conductive enclosure. The conductive enclosure functions to shield the electronics from electromagnetic interference. Thus, eddy current from MRIs may be reduced by reducing the size of the conductive enclosure, which may be accomplished by only encasing circuitry that needs to be protected from the external fields. The coil is not within the conductive enclosure, so that it can be used to perform charging and communication functions using an electromagnetic field. The non-conductive housing may include silicone or an epoxy. The non-conductive housing may be a poor conductor of both electricity and heat. Thus, the non-conductive housing does not generate the eddy currents that heat the device when in the presence of an electromagnetic field. Also, the non-conductive housing is a poor conductor of heat, and thus insulates the tissue from any heat generated at the coil or the conductive enclosure. The housing may be flexible, allowing some motion or flex when implanted, which encourages a low profile as it follows a curvature of a cranium when subcutaneously implanted over the cranium.

The illustrated housing may include a first housing portion and a second housing portion, where the first housing portion encapsulates the coil and the second housing portion encapsulates the conductive housing (e.g., metal can). The first and second housing portions may have substantially equal footprints. Each of the first and second housing portions have a thickness, length and width. The thickness may be uniform and may be less than the length and the width. Each of the first and second housing portions may have a substantially planar major surface, wherein the first and second housing portions are joined such that the substantially planar major surfaces form an angle between 90 degrees and 180 degrees. This angle allows the substantially planar major surfaces for the first and second housing portions to follow the curvature of the cranium. Another lobe may be included, which may encourage the implanted device to remain in place when implanted as it provides the implantable device with a profile that follows the curvature of the cranium.

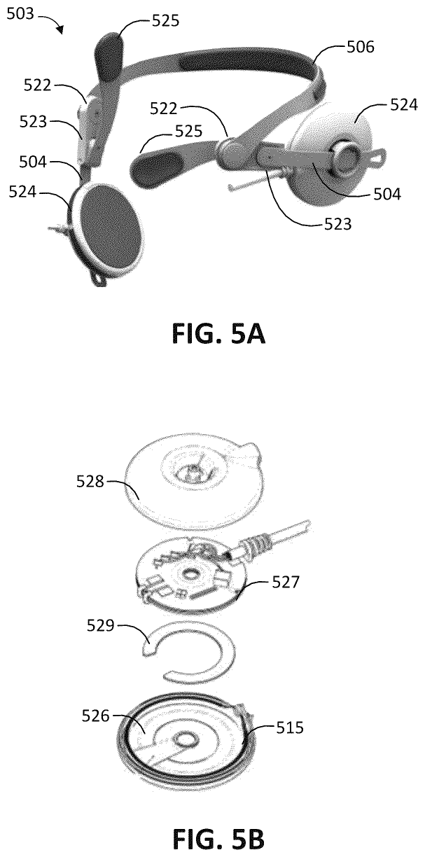

FIGS. 5A-5B illustrate a headset 503, similar to the headset illustrated in FIGS. 1A-1B. The headset 503 may include an adjustable frame 504 on each side of a main headset frame 506. The adjustable frames 504 are rotatably connected at pivot points 522 to the main headset frame 506. Each adjustable frame may include a secondary pivot point 523 to enable further adjustment of the coil enclosure 524 for placement over the head-located implanted devices. A bowed end 525 of the adjustable frames, opposite to the end with the coil enclosure 524, may be configured with bend inward to gently press against the head.

Each coil enclosure 524 may include a bottom 526 configured to hold the coil 515, a printed circuit board assembly (PCBA) 527, and a cover 528. An insulator gasket 529 may be positioned between the coil 515 and the PCBA 527. One or more sensors used for detecting temperature may be included in each coil enclosure 524. For example, a sensor may be included on a side of the PCBA 527 nearest the bottom 526 of the enclosure toward the patient's head. The headset may include sensor(s) in other locations. Some embodiments may use only sensor(s) in the headset to detect a temperature event. Some embodiments may use sensor(s) in the headset in conjunction with sensor(s) in the implantable device to detect a temperature event. Some embodiments may use only sensor(s) in the implantable medical device to detect a temperature event.

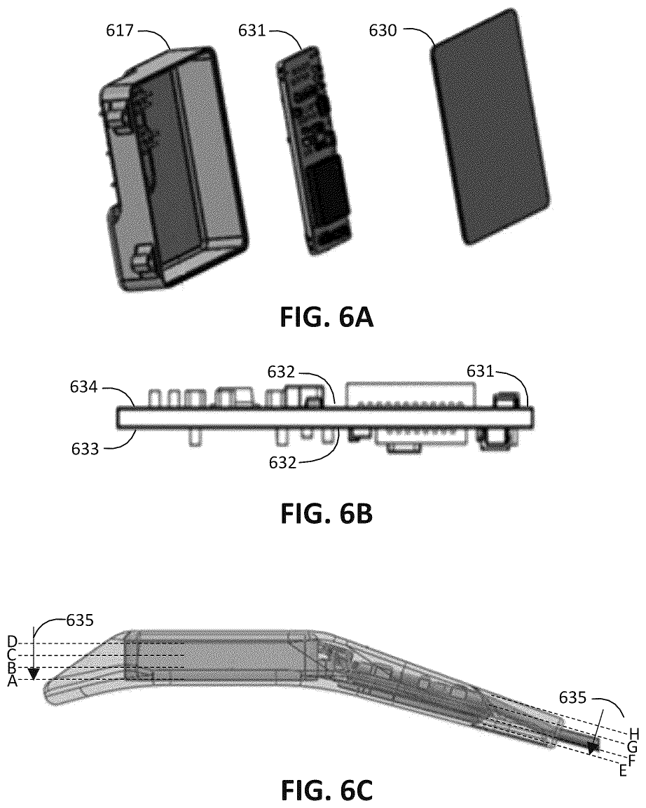

FIGS. 6A-6C illustrate, by way of example and not limitation, some components of an implantable device, including a conductive enclosure 617 (e.g., “can”) and lid 630 for encasing electronic circuitry (e.g., PCBA) 631. The implantable device may include sensor(s) 632 configured for use in detecting temperature event(s). The device is constructed such that the device enclosure 617 (“e.g., metal can”) and lid 630 encase the electronics 631 (PCBA) with sensor(s) 632 configured for use in detecting temperature event(s). Sensors can be placed on a top side 633 and/or bottom side 634 of the PCBA 631. Sensor(s) may also be placed on the conductive enclosure 617 and/or lid. The housing includes a first housing portion and a second housing portion. The thickness is less than the length and the width to provide each of the first and second housing portions with a substantially planar major surface, wherein the first and second housing portions are joined such that the substantially planar major surfaces form an angle between 90 degrees and 180 degrees. FIG. 6C generally illustrates, by way of example, and not limitations, four planes for each of the first and second housing portions. The planes may be parallel to the major surface of the housing portions, as generally illustrated in FIG. 6C (see planes A-D and planes E-H), and the major surfaces may be generally tangential to the curvature of the cranium. Sensor(s) may be positioned to directly measure temperature at different layers/planes. For example, a thermal model may be used to relate the output of multiple sensors using physical and thermal conductivity characteristics between sensors. Such models or algorithms may be used to predict the relationships between the sensors and the external temperatures, which may be used to determine temperature events and/or perform sensor diagnostics. Some embodiments may determine temperature gradients 635 using the sensors on different planes. Although the gradient is illustrated generally perpendicular to the major surface of the device, those of ordinary skill in the art would understand, upon reading and comprehending this disclosure, that the gradient may be in other directions and may depend on the specific locations of the temperature measurements. These “layered” temperature measurements may be used to ensure that neither the top or bottom of device is exceeding temperature limits.

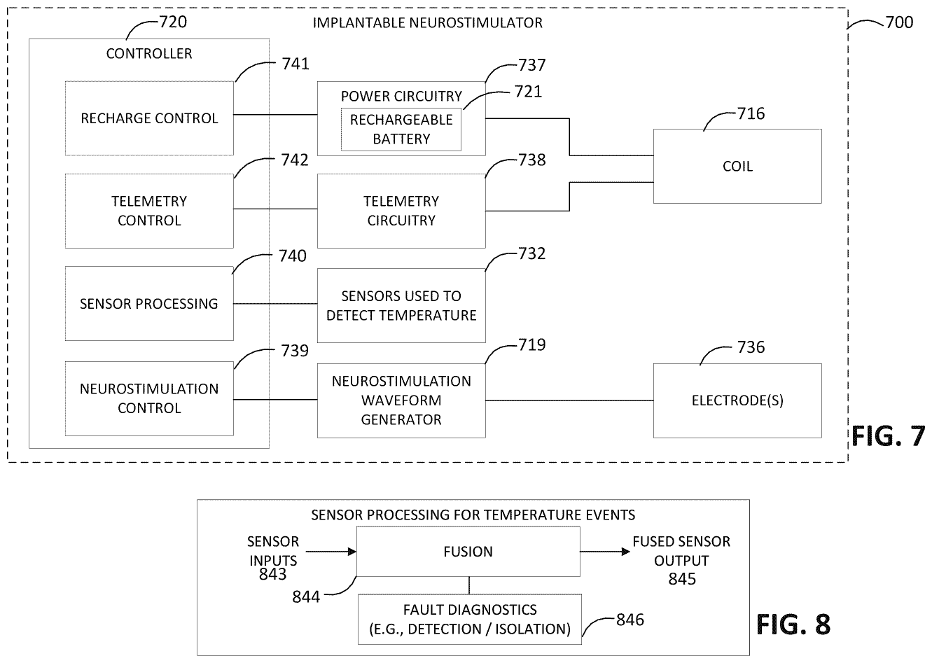

FIG. 7 illustrates, by way of example and not limitation, a diagram of a rechargeable implantable neurostimulator. The neurostimulator 700 may be configured for subcutaneous implantation and for managing heat during recharge. The neurostimulator 700 may include a neurostimulation waveform generator 719 configured to generate neurostimulation signals. The neurostimulation waveform generator 719 may be configured to generate pulses or trains of pulses using one or more pulse amplitudes, one or more pulse widths, and one or more pules frequencies. The trains of pulses may be delivered for various durations and according to various ON/OFF duty cycles. The pulses may be generally rectilinear or may have non-rectilinear shape. The neurostimulator 700 may include at least one electrode 736. The electrode(s) may be on an exterior of the implantable neurostimulator or may be on at least one stimulation lead. Some embodiments have more than one lead, where each lead may provide a plurality of electrodes capable of being selected for activation as an anode or cathode. The neurostimulator 700 may include at least one rechargeable battery 721 configured to power the rechargeable implantable neurostimulator, and a coil 716 for receiving power. The coil 716 is electrically connected to power circuitry 737 for use in recharging the rechargeable battery using the power received by the coil. The neurostimulator 700 may include telemetry circuitry 738 for use to send and receive telemetry via the coil 716. The neurostimulator 700 may include two or more sensors 732 configured for use in determining temperature. The sensors 732 may include temperature sensors configured to output a signal indicative of temperature, or may include other sensors configured to output a signal indicative of another condition based on temperature which can be used to determine temperature. The neurostimulator 700 may include a controller 720 configured to control generation of neurostimulation signals 739 from the neurostimulation waveform generator to deliver neurostimulation using the at least one electrode 736, perform sensor processing 740 using the two or more sensors 732 to determine a temperature event, and control recharging 741 of the rechargeable battery 721 using a recharging process, including modify the recharging process to reduce heating in response to determining that the temperature event occurred. The controller 720 may provide telemetry control 742. The controller 720 may be configured to determine whether the temperature event occurs by measuring implant temperature from multiple sensor readings and validate sensor measurements. The controller 720 may be configured to determine a sensor fault using the sensor readings, and adjust the sensor processing to account for the sensor fault when determining the temperature event.

FIG. 8 illustrates, by way of example and not limitation, sensor processing for temperature events performed using the controller illustrated in FIG. 7. The controller may receive sensor inputs 843, fuse the sensor inputs using a fusion process 844, and produce a fused sensor output 845 indicative of a temperature event. Fault diagnostics 846 may be used to adjust the fusion process for fusing the sensor inputs.

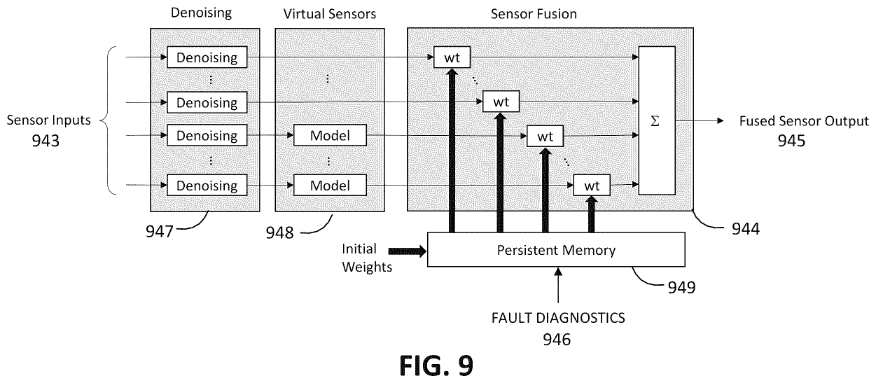

FIG. 9 illustrates, by way of example and not limitation, a sensing processing embodiment that weights sensor signals and uses fault diagnostics to adjust the weighted sensor signals. The illustrated embodiment includes a plurality of sensor inputs 943. The illustrated embodiments denoises the sensor inputs 947, may apply virtual sensors 948 to at least some of the signals using, by way of example, models, and performs a sensor fusion 944 that weights signals corresponding to each of the sensor inputs.

Denoising is a process of removing noise from a signal. Denoising may include applying band-pass filters, median filters, and autoregressive-moving average models to remove unwanted portions of the signal or noise. A virtual sensor may be a routine, which may but does not necessarily include a model, that converts a sensor measurement to a different scientific unit of measure.

Weighted sensor signals may be summed to provide a fused sensor output 945, which may be indicative of a temperature event. The weights (e.g., initial weights) for each signal may be stored in persistent memory 949, and fault diagnostics 946 may be used to adjust the weights for the signals. A simple implementation of sensor fusion is averaging. Enhanced sensor fusion may employ statistical-based methods such as Bayesian inferencing or Kalman Filtering when the uncertainty of each sensor is known a-priori. Similar uncertainty information can be obtained as part of the sensor fault detection and severity.

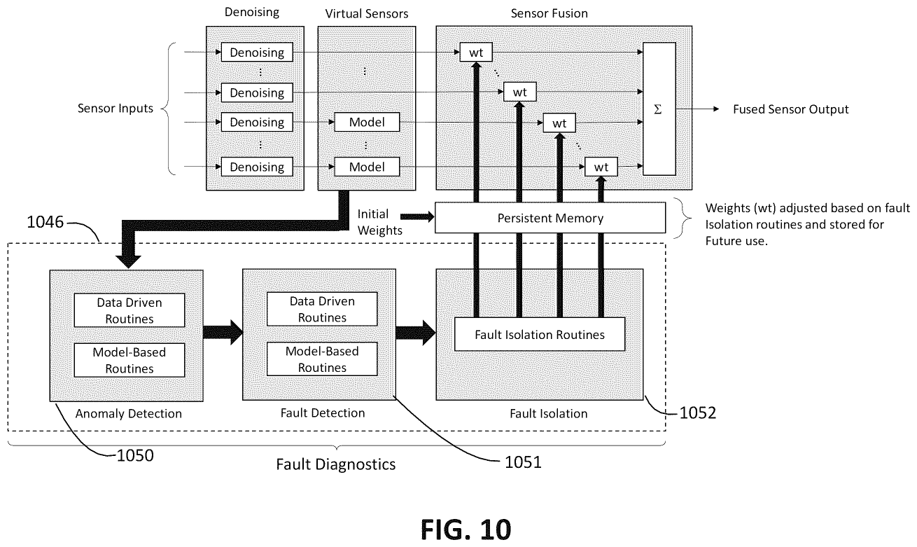

FIG. 10 illustrates, by way of example and not limitation, anomaly detection, fault detection and fault isolation used to weight sensor inputs. The illustrated fault diagnostics 1046 includes anomaly detection 1050, fault detection 1051 and fault isolation 1052. Fault diagnostics is a process of detecting a fault and isolating the source of the fault. For example, a backup sensor may be used if one or more sensors are identified as being faulty. In some embodiments, a weighted average of each sensor may be used such that the contribution of each sensor is dependent on the fault severity. Changing a weighted average between two sensors from 1 to 0 and 0 to 1 is equivalent of not using the sensor effectively resulting in the special cause of using a backup sensor. Such fault diagnostics along with sensor fusion may be used to ensure continuous operation in the event of a fault. The anomaly detection 1050 may be driven by data driven routines or model-based routines. Signals corresponding to the sensor inputs (e.g., form the virtual sensors), may be evaluated to detect anomalies in the signals. Anomaly detection detects data that significantly differs from a pattern. Anomaly detection may be periodically performed in the background to continuously monitor for occurrences of anomalies. Anomalies may be assessed through a combination of data driven methods and physics-based modeling. Data-driven anomaly detection methods may be based on expected behavior gained from historical data and a-priori information. Historical data may provide a baseline of expected behavior derived from previously collected data over a range of operating conditions using statistical measures such as computed means, standard deviations, outliers, repeated values, missing values, and noise. A-priori information may be acquired through expected behavior that can be derived from specifications such as operating limits and response times. Examples of data-driven anomaly detection may include, but is not limited to, the detection of: an occurrence rate of outliers, a positive trend in the mean occurs over time, spurious noise, and sensor outputs that deviate from other redundant sensors. Model-based anomaly detection methods may be based on the use of physical models used to compare two or more inputs. The illustrated fault detection 1051 may be data driven routines or model-based routines. Fault isolation routines 1052 may be performed to modify the weighting for the sensor inputs based on detected fault(s) from the fault detection 1051. An assessment of the fault based on the relative contribution of each sensor, may be referred to as a fault isolation. Fault isolation routines may be triggered when a fault has been declared during fault detection. The fault severity may be categorical or numerical. For example, a fault severity may correspond to a numerical scale from 0 to 1 where 0 corresponds to no fault and 1 corresponds to a fault.

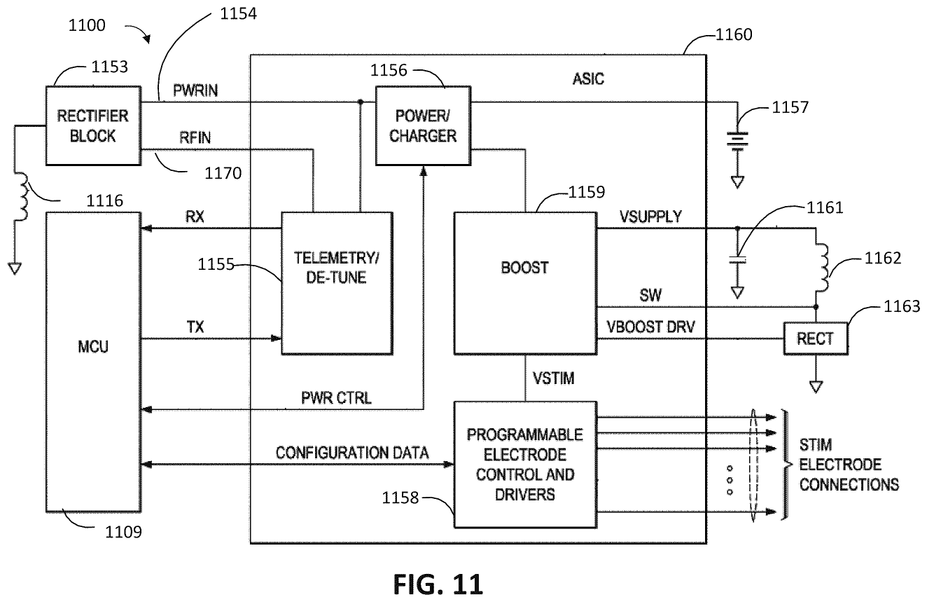

FIG. 11 illustrates, by way of example and not limitation, a block diagram of an implantable device 1100. A coil 1116 is connected to a rectifier Hock 1153 that generates a PWRIN signal 1154 and an RFIN signal 1170. Both the PWRIN signal 1154 and the REIN signal 1154 are connected to a telemetry/de-tune block 1155 that receives a forward telemetry signal (REIN signal 1154), and which interacts with the PWRIN de-tunes the coil 1116 to thereby communicate back telemetry information and/or disable further energy transfer to the coil 1116. The PWRIN signal 1154 is received by a powerlcharger block 1156 receives the PWRIN signal 1154 to generate one or more internal voltages for circuitry of the implantable device 1100, and for charging battery 1157.

A microcontroller (MCU) 1109 provides overall configuration. and communication functionality and communicates forward telemetry (RX signal) and back telemetry (TX signal) information via a pair of data lines coupled to the telemetry block 1155. The MCU 1109 receives information from and provides configuration information to/from the power/charger block 1156 via control signals PWR CTRL. A programmable electrode control and driver block (drivers) 1158 generates electrical stimulation signals on each of a group of individual electrodes. An adjustable voltage generator circuit boost 1159, which is coupled via signals VSUPPLY, SW. and VBOOST DRV to components external to the ASIC 1160 (including capacitor 1161, inductor 1162, and rectifier block 1163) provides a power supply voltage VSTIM to the drivers block 1158.

The MCU 1109 provides configuration information to the drivers block 1158 via configuration signals CONFIGURATION DATA. In some embodiments, the power charger block 1156, the telemetry block 1155, the boost circuit 1159, and the drivers block 1158 are all implemented in a single application specific integrated circuit (ASIC) 1160, although such is not required. In the overall operation, the ASIC 1160 may function as a state machine that operates independently of the MCU 1109. The MCU 1109 may include nonvolatile memory for storing configuration data from the external control system to allow a user to download configuration data to the MCU 1109. The MCU 1109 may then transfer this configuration data to ASIC 1160 in order to configure the state machine therein. In this manner, the MCU 1109 does not have to operate to generate the driving signals on the electrodes which may reduce the power requirements. Other embodiments may implement one or more of these three functional blocks using a combination of multiple ASIC's, off-the-shelf integrated circuits, and discrete components.

Battery charging (charge delivery) may be monitored and adjusted to provide the most efficient charging (charge delivery) conditions, limit unnecessary power dissipation, and address temperature conditions. Preferable conditions for charging the battery may include a charging voltage of approximately 4.5 V for most efficient energy transfer (with a minimum charging voltage of about 4.0 V). Also, it is particularly desirable to maintain a constant charging current into the battery in a battery charging operation during the entire charging time, even as the battery voltage increases as it charges. Preferably this constant charging current is about C/2, which means a charging current that is one-half the value of the theoretical current draw under which the battery would deliver its nominal rated capacity in one hour. To accomplish this, a variety of sensors and monitors (not shown) may be included within the device 1100 to measure power levels, voltages (including the battery voltage itself), charging current, and one or more internal temperatures.

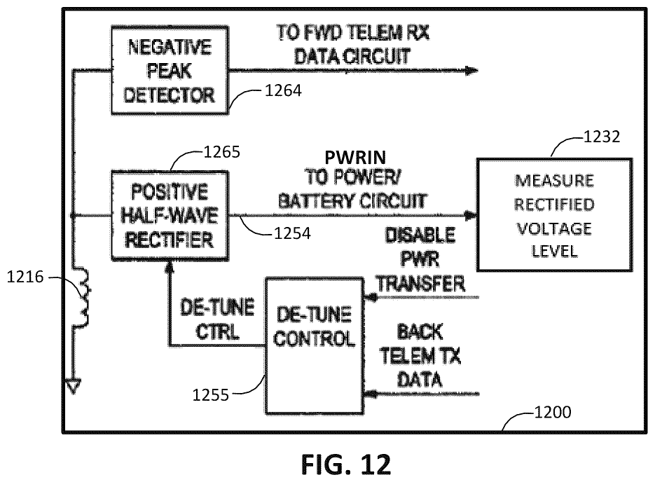

FIG. 12 illustrates, by way of example and not limitation, an embodiment of an implantable device. The implantable device of FIG. 7 may be a more specific example of the device illustrated in FIG. 6. The illustrated implantable device 1200 includes a coil 1216, a negative peak detector 1264, and a positive half-wave rectifier 1265. The coil 1216 may be coupled via node to a negative peak detector block 1264 for receiving forward telemetry data and generating a respective forward telemetry receive data signal. The coil 1216 coupled to the positive half-wave rectifier Hock 1265 receives energy and generates a rectified voltage (PWRIN 1254), which may be provided to a power/battery circuit. The illustrated device 1200 may include a sensor 1232 configured to measure the rectified voltage level of PWRIN 1254. The device 1200 may be configured to communicate data corresponding to the measured PWRIN 1254 to the external device, which may use this data to control the charging and alignment routines. The positive half-wave rectifier block 1265 may be responsive to a DE-TUNE signal for de-tuning the coil 1216 to inhibit transfer of energy from the external device, The implantable device may also include a de-tune control block 1255 for generating the DE-TUNE control signal responsive to a disable power transfer signal DISABLE PWR TRANSFER, and/or responsive to a bit-serial back telemetry transmit data signal BACK TELEM TX DATA. In operation, the DISABLE PWR TRANSFER signal may he asserted when charging (or charge transfer) is complete or not desired such as in the event of a detected temperature event. The DISABLE PWR TRANSFER signal asserts the DE-TUNE control signal to de-tune the receive coil through the positive half-wave rectifier. Detuning may also be used for communication. During normal charging the DE-TUNE control signal may be asserted for each bit-position of the bit-serial BACK TELEM TX DATA signal corresponding to one of its two data states. Since de-tuning the positive half-wave rectifier in concert with the receive coil inhibits energy transfer from the transmit coil to the receive coil, the loading of transmit coil is decreased. In the external device, the receiver circuit senses the change in peak current through the corresponding transmit coil as each serial data bit of the BACK TELEM TX DATA signal either tunes or de-tunes the receive coil, and generates accordingly a back telemetry receive data signal BACK TELEM RX DATA. If the DE-TUNE control signal is already asserted (e.g., because the DISABLE PWR TRANSFER signal is asserted to indicate charging/charge transfer is complete or not desired) when the charge receiving system desires to transmit back telemetry data, the DISABLE PWR TRANSFER signal may be briefly de-asserted to allow the BACK TELEM TX DATA signal to control the DR-TUNE control signal. Thus, the charge receiving system may still transmit back telemetry information irrespective of whether it is generally in a de-tuned state.

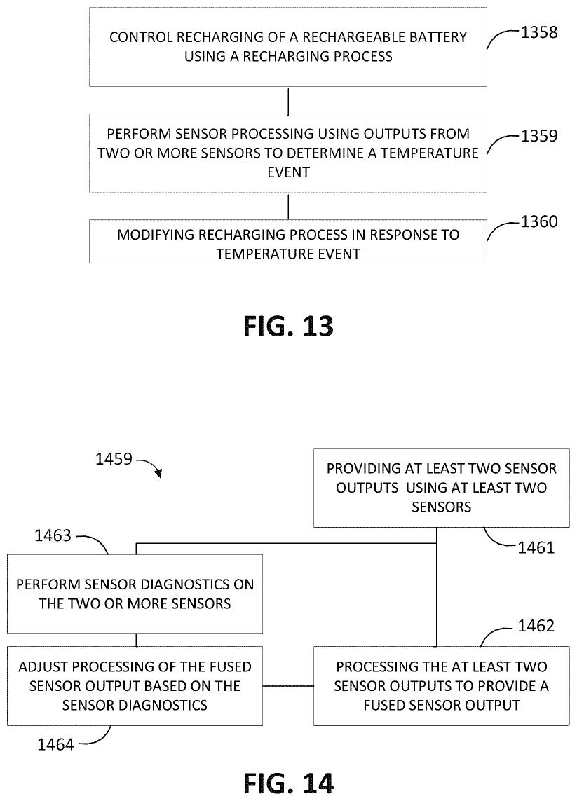

FIG. 13 illustrates, by way of example and not limitation, a method for managing heat during recharge of a rechargeable battery in an implantable medical device. The method may include, at 1358, controlling recharging of the rechargeable battery using a recharging process. The method may further include, at 1359, performing sensor processing using outputs from two or more sensors to determine a temperature event. The method may further include, at 1360, modifying the recharging process to reduce heating in response to determining that the temperature event occurred. The sensor processing may be performed by receiving two or more signals corresponding to the two or more sensors and producing a fused sensor output using the two or more signals. The fused sensor output may be indicative of whether the temperature event occurred. The method may include denoising the received two or more signals, applying a model to at least one of the received two or more signals to provide a virtual sensor signal used to produce the fused sensor output, weighting the received two or more signals to produce the fused sensor output, and performing sensor diagnostics. The method may include adjusting production of the fused sensor output based on the performed sensor diagnostics. The sensor diagnostics may include an isolation routine to remove one or more of the two or more signals from being used to produce the fused sensor output. The sensor diagnostics may include an isolation routine to reduce a weight for one or more of the two or more signals when used to produce the fused sensor output. Some embodiments may modify the recharging process in response to the temperature event by providing a signal to an external device to stop the external device from recharging the neurostimulator. Some embodiments may modify the recharging process in response to the temperature event by detuning the implantable neurostimulator to reduce heat by lowering ohmic losses generated on the coil.

FIG. 14 illustrates, by way of example and not limitation, a method for performing sensor processing using outputs from two or more sensors. The process 1459 may be an example of performing sensor processing 1359 in FIG. 13. At 1461, at least two sensor outputs are provided using at least two sensors. One or more of the sensors may sense temperature. One or more of the sensors may sense another parameter from which temperature may be derived using algorithms or models. At 1462, the at least two sensor outputs are processed to provide a fused sensor output. The fused sensor output may be indicative of whether a temperature event has occurred. For example, a temperature event may be deemed to have occurred if the fused sensor output is at or above a threshold, or alternatively at or below the threshold. Sensor diagnostics may be performed on the two or more sensors at 1463, and at 1464 adjustments to the processing of the fused sensor output may be determined based on the sensor diagnostics. The determined adjustments may be implemented in the processing illustrated at 1462. The resulting fused sensor output is fault tolerant because of the use of the sensor diagnostics to adjust the processing.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A rechargeable implantable neurostimulator for subcutaneous implantation and for managing heat during recharge, comprising:
a neurostimulation waveform generator configured to generate neurostimulation signals;
at least one electrode;
at least one rechargeable battery configured to power the rechargeable implantable neurostimulator;
a coil for receiving power, wherein the coil is electrically connected to the circuitry for use in recharging the rechargeable battery using the power received by the coil;
two or more sensors configured for use in determining temperature;
a controller configured to:
control generation of neurostimulation signals from the neurostimulation waveform generator to deliver neurostimulation using the at least one electrode;
perform sensor processing using the two or more sensors to determine a temperature event; and
control recharging of the rechargeable battery using a recharging process, including modify the recharging process to reduce heating in response to determining that the temperature event occurred.

2. The rechargeable implantable neurostimulator of claim 1, wherein the controller is configured to determine whether the temperature event occurs by measuring implant temperature from multiple sensor readings, and validating sensor measurements.

3. The rechargeable implantable neuro stimulator of claim 1, wherein the controller is configured to determine a sensor fault using the sensor readings, and adjust the sensor processing to account for the sensor fault when determining the temperature event.

4. The rechargeable implantable neurostimulator of claim 1, wherein the two or more temperature sensors are positioned to detect temperature at different depths from tissue when the neurostimulator is subcutaneously implanted.

5. The rechargeable implantable neuro stimulator of claim 1, further comprising a housing for housing at least one of the neurostimulation waveform generator, the battery, the coil or the controller, wherein the two or more temperature sensors include an external temperature sensor configured to sense a temperature outside of the housing and an internal temperature sensor configured to sense a temperature inside of the housing.

6. The rechargeable implantable neurostimulator of claim 1, wherein the two or more temperature sensors include a same type of temperature sensor.

7. The rechargeable implantable neuro stimulator of claim 1, wherein the two or more temperature sensors include a different type of temperature sensor.

8. The rechargeable implantable neuro stimulator of claim 1, wherein the controller is configured to perform sensing processing by receiving two or more signals corresponding to the two or more sensors, and producing a fused sensor output using the two or more signals, wherein the fused sensor output is indicative of whether the temperature event occurred.

9. The rechargeable implantable neurostimulator of claim 8, wherein the controller is configured to denoise the received two or more signals, and apply a model to at least one of the received two or more signals to provide a virtual sensor signal used to produce the fused sensor output.

10. The rechargeable implantable neurostimulator of claim 8, wherein the controller is configured to weight the received two or more signals to produce the fused sensor output.

11. The rechargeable implantable neurostimulator of claim 8, wherein the controller is configured to perform sensor diagnostics and adjust production of the fused sensor output based on the performed sensor diagnostics.

12. The rechargeable implantable neurostimulator of claim 11, wherein the sensor diagnostics include an anomaly detection process or a fault detection process, and the sensor diagnostics further include an isolation routine to:
remove one or more of the two or more signals from being used to produce the fused sensor output; or
reduce a weight for one or more of the two or more signals when used to produce the fused sensor output.

13. The rechargeable implantable neurostimulator of claim 1, wherein the controller is configured to modify the recharging process in response to the temperature event by:
providing a signal to an external device to stop the external device from recharging the neurostimulator; or
detuning the implantable neurostimulator to reduce an amount of received energy to be dissipated as heat when the at least one rechargeable battery is fully charged.

14. The rechargeable implantable neurostimulator of claim 1, further comprising:
a metal can configured to house the neurostimulator waveform generator and the controller, wherein the metal can is configured to shield the neurostimulator waveform generator and the controller from electromagnetic interference and moisture ingress, and the coil is biocompatible and not housed within the metal can; and
a housing configured to encapsulate the coil and the metal can, wherein the housing is non-conductive and biocompatible.

15. The rechargeable implantable neurostimulator of claim 14, wherein the housing includes silicone or an epoxy.

16. The rechargeable implantable neurostimulator of claim 14, wherein the housing is a flexible housing.

17. The rechargeable implantable neurostimulator of claim 14, wherein:
the housing includes a first housing portion and a second housing portion;
the first housing portion encapsulates the coil and the second housing portion encapsulates the metal can;
the first and second housing portions have substantially equal footprints; and
each of the first and second housing portions have a thickness, length and width, the thickness is less than the length and the width to provide each of the first and second housing portions with a substantially planar major surface, wherein the first and second housing portions are joined such that the substantially planar major surfaces form an angle between 90 degrees and 180 degrees.

18. A method for managing heat during recharge of a rechargeable battery in an implantable medical device, the method comprising:
controlling recharging of the rechargeable battery using a recharging process;
performing sensor processing using outputs from two or more sensors to determine a temperature event; and
modifying the recharging process to reduce heating in response to determining that the temperature event occurred.

19. The method of claim 18, wherein the sensor processing is performed by receiving two or more signals corresponding to the two or more sensors, and producing a fused sensor output using the two or more signals, wherein the fused sensor output is indicative of whether the temperature event occurred.

20. The method of claim 19, further comprising:
denoising the received two or more signals;
applying a model to at least one of the received two or more signals to provide a virtual sensor signal used to produce the fused sensor output;
weighting the received two or more signals to produce the fused sensor output; and
performing sensor diagnostics and adjust production of the fused sensor output based on the performed sensor diagnostics.

21. The method of claim 20, wherein the sensor diagnostics include an isolation routine to:
remove one or more of the two or more signals from being used to produce the fused sensor output; or
reduce a weight for one or more of the two or more signals when used to produce the fused sensor output.

22. The method of claim 20, further comprising modifying the recharging process in response to the temperature event by:
providing a signal to an external device to stop the external device from recharging the neurostimulator; or
detuning the implantable device to reduce an amount of received energy to be dissipated as heat when the at least one rechargeable battery is fully charged.

23. The method of claim 18, wherein the temperature event is determined before initiating recharging of the rechargeable battery.

24. The method of claim 18, further comprising receiving user input used to program a temperature threshold for patient comfort, the temperature event is determined using the programmed temperature threshold.

25. A system, comprising:
a rechargeable implantable neurostimulator for subcutaneous implantation and for managing heat during recharge, the rechargeable implantable neurostimulator comprising:
a neurostimulation waveform generator configured to generate neurostimulation signals;
at least one electrode;
at least one rechargeable battery configured to power the rechargeable implantable neurostimulator; and
a coil for receiving power, wherein the coil is electrically connected to the circuitry for use in recharging the rechargeable battery using the power received by the coil; and
an external device configured to wirelessly charge and communicate with the rechargeable implantable stimulator;
wherein the rechargeable implantable neurostimulator includes at least one sensor for use in determining temperature, and the rechargeable implantable neurostimulator includes at least one sensor for use in determining temperature, and wherein the system is configured to perform sensor processing using the two or more sensors to determine a temperature event, and control recharging of the rechargeable battery using a recharging process, including modify the recharging process to reduce
heating in response to determining that the temperature event occurred.
